SEQUENCE LISTING (6 sequences)

SEQ ID NO: 1 - protein, 520 aa, Pseudomonas aeruginosa
MKPEDFRASATRPFTGEEYLASLRDDREIYIYGDRVKDVTSHPAFRNAAASMARLYDALHDPQSKEKLCWETDTGNGGYTHKFFRYARSADELRQQRDAIAEWSRLTYGWMGRTPDYKAAFGSALGANPGFYGRFEDNAKTWYKRIQEACLYLNHAIVNPPIDRDKPVDQVKDVFISVDEEVDGGIVVSGAKVVATNSALTHYNFVGQGSAQLLGDNTDFALMFIAPMNTPGMKLICRPSYELVAGIAGSPFDYPLSSRFDENDAILVMDKVFIPWENVLIYRDFERCKQWFPQGGFGRLFPMQGCTRLAVKLDFITGALYKALQCTGSLEFRGVQAQVGEVVAWRNLFWSLTDAMYGNASEWHGGAFLPSAEALQAYRVLAPQAYPEIKKTIEQVVASGLIYLPSGVRDLHNPQLDKYLSTYCRGSGGMGHRERIKILKLLWDAIGSEFGGRHELYEINYAGSQDEIRMQALRQAIGSGAMKGMLGMVEQCMGDYDENGWTVPHLHNPDDINVLDRIRQ

SEQ ID NO: 2 - protein, 170 aa, Pseudomonas aeruginosa
MSQLEPRQQAFRNAMAHLSAAVNVITSNGPAGRCGITATAVCSVTDSPPTLMLCINRNSEMNTVFKANGRLCVNVLSGEHEEVARHFAGMTEVPMERRFALHDWREGLAGLPVLHGALANLQGRIAEVQEIGTHSVLLLELEDIQVLEQGDGLVYFSRSFHRLQCPRRAA

SEQ ID NO: 3 - DNA, 30 bp, Pseudomonas aeruginosa
ttctcatgaaacccgaagatttccgtgcct

SEQ ID NO: 4 - DNA, 30 bp, Pseudomonas aeruginosa
cgcggatcctcattggcggatgcgatcgag

SEQ ID NO: 5 - DNA, 30 bp, Pseudomonas aeruginosa
ttccatatgtcccagctcgaacccaggcag

SEQ ID NO: 6 - DNA, 30 bp, Pseudomonas aeruginosa
ttcggtacctcaggccgcccgccgggggca

(19) United States
(12) Patent Application Publication
(10) Pub. No.: US 2017/0037437 A1
(43) Pub. Date: Feb. 9, 2017

(54) HYDROXYSTILBENE PRODUCTION METHOD

(71) Applicant: MORINAGA & CO., LTD., Tokyo (JP)

(72) Inventors: Kuniki KINO, Tokyo (JP); Toshiki FURUYA, Tokyo (JP)

(21) Appl. No.: 15/110,850

(22) PCT Filed: Jan. 6, 2015
(86) PCT No.: PCT/JP2015/050177

§ 371 Date: Oct. 17, 2016

(30) Foreign Application Priority Data
Jan. 10, 2014 (JP) 2014-003638

(51) Int. Cl. C12P 7/22

(52) U.S. Cl. CPC C12P 7/22; C12Y 114/13

(57) ABSTRACT

The present invention is directed to provide novel methods of producing hydroxystilbene. According to the present invention, as a method of producing a compound having the general formula (II),

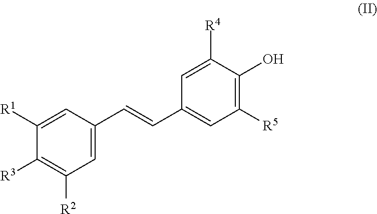

in which R¹ to R³ are each —H, —OH or —OR; R is independently selected from a C1-6 alkyl group and a glycosyl group; and one of R⁴ and R⁵ is —H and the other is —OH, 4-hydroxyphenylacetate 3-hydroxylase (HPAH) is administered to a compound having the general formula (I),

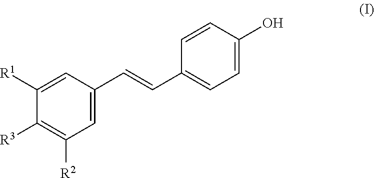

in which R¹ to R³ are each —H, —OH or —OR; and R is independently selected from a C1-6 alkyl group and a glycosyl group.

### TECHNICAL FIELD

The present invention relates to methods of producing hydroxystilbene.

### BACKGROUND ART

Piceatannol (i.e., 3′,4′,3,5-tetrahydroxy-trans-stilbene or 3,3′,4′,5-tetrahydroxy-trans-stilbene) having the structure of resveratrol which has been hydroxylated at the 3′-position is a notable compound that has been shown to exhibit various physiological activities such as anti-oxidative activity and anti-cancer activity (Mutat Res-Fund Mol M vol. 750, p. 60-82 (2012)). Focusing on the various physiological activities of piceatannol, extracts of the passion fruit seeds containing the compound have been investigated for the purpose of healthcare and medical applications (JP-A-2010-030911, JP-A-2013-151457, and JP-A-2013-151458).

Although piceatannol is contained in plants such as passion fruits and grapes, its extraction from the plants is not efficient at all because the content is small (WO2010/113315). Piceatannol can also be obtained by organic synthesis but many complicated reaction procedures are required.

It has recently been disclosed that 4-hydroxyphenylacetate 3-hydroxylases (HPAHs; EC 1.14.13.3) (synonym: p-hydroxyphenylacetate 3-hydroxylase, 4-hydroxyphenylacetate 3-monooxygenase, p-hydroxyphenylacetate hydroxylase, 4-HPA 3-hydroxylase) from Escherichia coli are capable of converting resveratrol into piceatannol (Curr Opin Biotech vol. 26 p. 71-78 (2014)). Yields are, however, still unsatisfactory.

### SUMMARY OF THE INVENTION

An object of the present invention is to provide novel methods of producing hydroxystilbene.

### Means to Solve the Problems

4-hydroxyphenylacetate 3-hydroxylases (HPAHs; EC 1.14.13.3) (synonyms: p-hydroxyphenylacetate 3-hydroxylase, 4-hydroxyphenylacetate 3-monooxygenase, p-hydroxyphenylacetate hydroxylase, 4-HPA 3-hydroxylase) are known to be two-component FAD-dependent monooxygenases and be capable of synthesizing ortho-diphenol compounds from monophenol compounds using flavin as a coenzyme.

The present inventors searched for an effective method of producing piceatannol and then found that HPAHs are capable of efficiently converting resveratrol into piceatannol. The present invention was thus completed.

An aspect of the present invention is a method of producing a compound having the general formula (II):

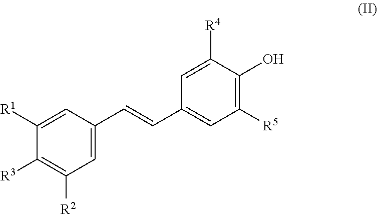

in which R¹ to R³ are each —H, —OH or —OR; R is independently selected from a C1-6 alkyl group and a glycosyl group; and one of R⁴ and R⁵ is —H and the other is —OH, including the step of administering 4-hydroxyphenylacetate 3-hydroxylase (HPAH) to a compound having the general formula (I):

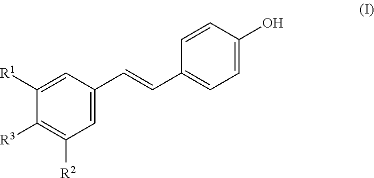

in which R¹ to R³ are each —H, —OH or —OR; and R is independently selected from a C1-6 alkyl group and a glycosyl group.

Another aspect of the present invention is a method of producing a compound having the general formula (III):

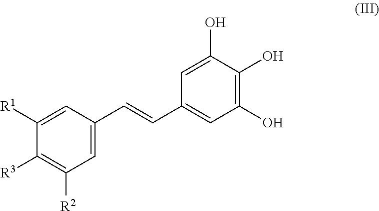

in which R¹ to R³ are each —H, —OH or —OR; and R is independently selected from a C1-6 alkyl group and a glycosyl group, including the step of administering 4-hydroxyphenylacetate 3-hydroxylase (HPAH) to a compound having the general formula (I):

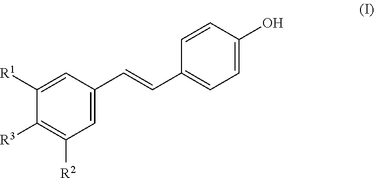

in which R¹ to R³ are each —H, —OH or —OR; and R is independently selected from a C1-6 alkyl group and a glycosyl group, and/or a compound having the general formula (II):

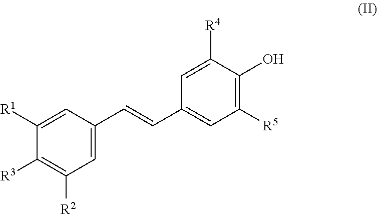

in which R¹ to R³ are each —H, —OH or —OR; R is independently selected from a C1-6 alkyl group and a glycosyl group; and one of R⁴ and R⁵ is —H and the other is —OH.

In either method described above, the compound having the general formula (II) may be piceatannol. The HPAH may be a recombinant protein or a natural protein.

In either method described above, a purified HPAH recombinant protein, a purified HPAH natural protein, a bacterium expressing an endogenous HPAH or an extract from the bacterium, or a microorganism expressing an exogenous HPAH or an extract from such microorganism may be administered to the compound that is a substrate. The bacterium expressing an endogenous HPAH may be Pseudomonas aeruginosa.

A further aspect of the present invention is a method of measuring an enzymatic activity to convert a compound having the general formula (I):

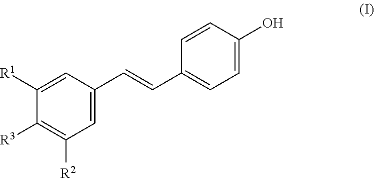

in which R¹ to R³ are each —H, —OH or —OR; and R is independently selected from a C1-6 alkyl group and a glycosyl group, into a compound having the general formula (II):

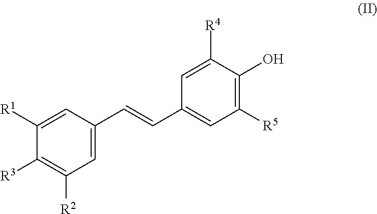

in which R¹ to R³ are each —H, —OH or —OR; R is independently selected from a C1-6 alkyl group and a glycosyl group; and one of R⁴ and R⁵ is —H and the other is —OH, including the step of administering a 4-hydroxyphenylacetate 3-hydroxylase (HPAH) analog to the compound having the general formula (I) to detect the compound having the general formula (II), the analog being a protein having an amino acid sequence which is at least 50% homologous to HpaB or HpaC derived from a Pseudomonas aeruginosa PAO1 strain.

A yet another aspect of the present invention is a method of screening 4-hydroxyphenylacetate 3-hydroxylase (HPAH) analogs for an enzyme capable of converting a compound having the general formula (I):

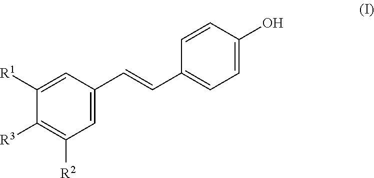

in which R¹ to R³ are each —H, —OH or —OR; and R is independently selected from a C1-6 alkyl group and a glycosyl group, the analog being a protein having an amino acid sequence which is at least 50% homologous to HpaB or HpaC derived from a Pseudomonas aeruginosa PAO1 strain, into a compound having the general formula (II):

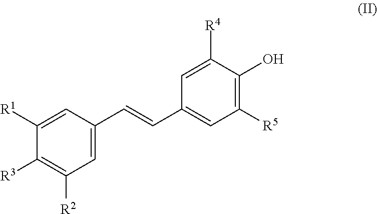

in which R¹ to R³ are each —H, —OH or —OR; R is independently selected from a C1-6 alkyl group and a glycosyl group; and one of R⁴ and R⁵ is —H and the other is —OH, including the steps of administering the 4-hydroxyphenylacetate 3-hydroxylase (HPAH) analogs to the compound having the general formula (I) to detect the compound having the general formula (II); and selecting an HPAH analog with which the compound having the general formula (II) is detected.

A still another aspect of the present invention is a method of measuring an enzymatic activity to convert a hydroxyphenyl compound as a substrate into a dihydroxyphenyl compound including the step of administering 4-hydroxyphenylacetate 3-hydroxylase (HPAH) to the hydroxyphenyl compound to detect the dihydroxyphenyl compound, the hydroxyphenyl compound being a compound having the general formula (I):

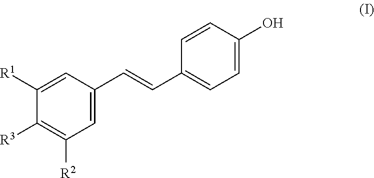

in which R¹ to R³ are each —H, —OH or —OR; and R is independently selected from a C1-6 alkyl group and a glycosyl group.

Another aspect of the present invention is a method of screening hydroxyphenyl compounds as a substrate of 4-hydroxyphenylacetate 3-hydroxylase (HPAH) that catalyzes conversion of a hydroxyphenyl compound into a dihydroxyphenyl compound including the steps of administering the 4-hydroxyphenylacetate 3-hydroxylase (HPAH) to the hydroxyphenyl compounds to detect the dihydroxyphenyl compound; and selecting the substrate of HPAH with which the dihydroxyphenyl compound is detected, the hydroxyphenyl compound being a compound having the general formula (I):

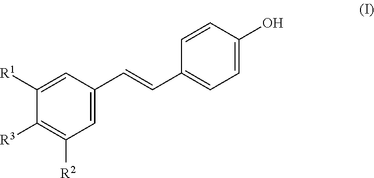

in which R¹ to R³ are each —H, —OH or —OR; and R is independently selected from a C1-6 alkyl group and a glycosyl group.

### BRIEF DESCRIPTION OF THE DRAWINGS

FIG. 1 is a schematic diagram illustrating (A) the mechanism of conversion and (B) the mechanism of conversion in the bacterial reaction system, through which HPAH converts a compound.

FIG. 2 is amino acid sequences of HpaBC derived from the Pseudomonas aeruginosa PAO1 strain.

FIG. 3 shows a result of HPLC analysis for the conversion of resveratrol by HpaBC-expressing recombinant Escherichia coli in an example of the present invention.

FIG. 4 is a graph showing a result of piceatannol synthesis by HpaBC-expressing recombinant Escherichia coli on a flask scale in an example of the present invention.

### EMBODIMENTS FOR CARRYING OUT THE INVENTION

Unless otherwise noted in embodiments and examples, all procedures used are according to standard protocols such as J. Sambrook, E. F. Fritsch & T. Maniatis (Ed.), Molecular cloning, a laboratory manual (3rd edition), Cold Spring Harbor Press, Cold Spring Harbor, N.Y. (2001); and F. M. Ausubel, R. Brent, R. E. Kingston, D. D. Moore, J. G. Seidman, J. A. Smith, K. Struhl (Ed.), Current Protocols in Molecular Biology, John Wiley & Sons Ltd., with or without modifications or changes. In addition, commercial reagent kits or measurement instruments are used as described in protocols attached thereto, unless otherwise noted.

The objects, features, advantages, and ideas of the present invention are apparent to those skilled in the art from the description of this specification. Those skilled in the art can easily reproduce the present invention from the description herein. The embodiments and specific examples described below represent preferable aspects of the present invention, which are given for the purpose of illustration or explanation. The present invention is not limited thereto. It is obvious to those skilled in the art that various changes and modifications may be made according to the description of the present specification within the spirit and scope of the present invention disclosed herein.

### ==Method of Converting Compounds==

According to an embodiment of the present invention, it is possible to synthesize the compound having the general formula (II) (hereinafter, referred to as compound (II)) by administering the enzyme HPAH to the compound having the general formula (I) (hereinafter, referred to as compound (I)). Furthermore, this reaction can be used to measure the activity of the HPAH enzyme that catalyzes conversion of the compound (I) into the compound (II).

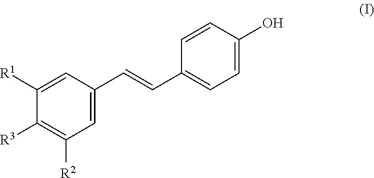

in which R¹ to R³ are each —H, —OH or —OR; and R is independently selected from a C1-6 alkyl group and a glycosyl group; and

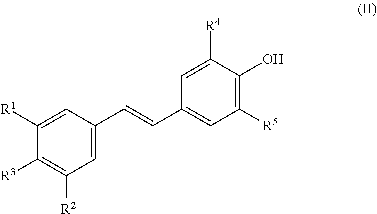

in which R¹ to R³ are each —H, —OH or —OR; R is independently selected from a C1-6 alkyl group and a glycosyl group; and one of R⁴ and R⁵ is —H and the other is —OH.

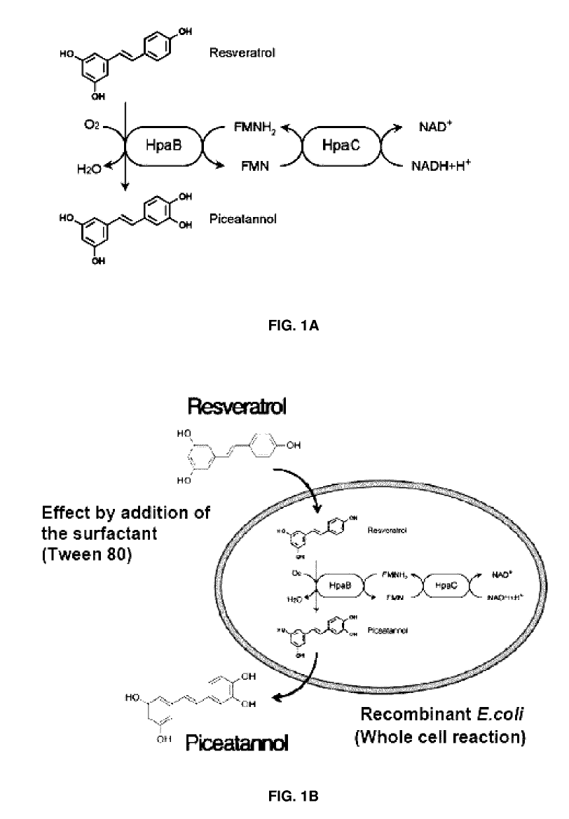

It is preferable that R³ is —H. Furthermore, it is preferable that both R¹ and R² are —OH or —OR, and R is independently selected from a C1-6 alkyl group and a glycosyl group. It is most preferable that both R¹ and R² are —OH; in this case, the compound (I) is resveratrol and the compound (II) is piceatannol. A mechanism of conversion activity of the enzyme during the conversion is shown in FIG. 1.

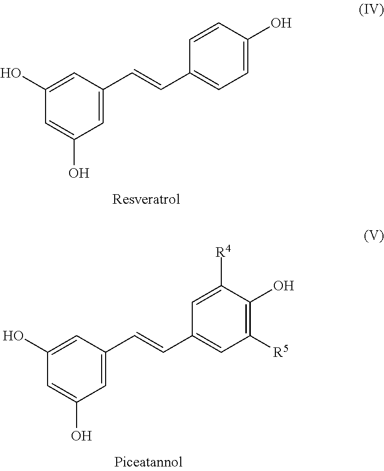

in which one of R⁴ and R⁵ is —H and the other is —OH.

Moreover, by administering HPAH for a long time, a compound having the general formula (III) (hereinafter, referred to as a compound (III)) can be synthesized from the compound (I) or the compound (II):

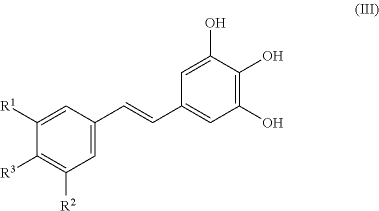

in which R¹ to R³ are each —H, —OH or —OR; and R is independently selected from a C1-6 alkyl group and a glycosyl group.

The two-component flavin-dependent oxygenase HPAH to be administered consists of HpaB which is a subunit of monooxygenase and HpaC which is a subunit of oxidoreductase. The source of HpaB and HpaC is not specifically limited and examples include bacteria classified into genera such as Pseudomonas, Escherichia, Enterobacter, Krebsiella, Salmonella, Serratia, Shigella, Yersinia, Rhizobium, Bradyrhizobium, Mesorhizobium, Sinorhizobium, Bacillus, Geobacillus, Saccharothrix, Streptomyces, Rhodococcus, Gordonia, Mycobacterium, Thermus, Acinetbacter, Halomonas, and Micrococcus. A pseudomonad such as Pseudomonas putida, Pseudomonas aeruginosa, and Pseudomonas ovalis is preferable, and Pseudomonas aeruginosa or Escherichia coli is particularly preferable. For Pseudomonas aeruginosa, a PAO1 strain is more preferable. For Escherichia coli, a W strain or a B strain is preferable. It should be noted that HpaB and HpaC herein include all homologs and mutants which are capable of functioning as a two-component flavin-dependent oxygenase when interacted with each other, unless the enzyme is specified based on a species. Each homolog or mutant may have a natural sequence or an artificial sequence. HpaB and HpaC may be derived from the same species or different species. At least one of them may have an artificial sequence. The homolog or mutant is a protein having an amino acid sequence which is at least 70%, preferably at least 80%, more preferably at least 90%, and yet more preferably at least 95% homologous to HpaB or HpaC of Pseudomonas aeruginosa. The method of measuring homology is not particularly limited and examples include BLAST, FASTA, and PSI-BLAST.

The type of HpaB and HpaC is not specifically limited. They may independently be a recombinant protein or a natural protein.

As way of example where HPAH is a recombinant protein, when the hpaB gene that encodes HpaB and the hpaC gene that encodes HpaC, which make up HPAH, are both introduced into a host microorganism and allowed to be expressed therein, HPAH is reconstructed in the recombinant microorganism. Since the compound (I) penetrates through the cell membrane, addition of the compound (I) to the medium in which the recombinant microorganism has been cultured results in synthesis of the compound (II) and/or the compound (III) in the cells. Accordingly, the recombinant microorganism itself can be used for the conversion of the compound. The host microorganism is not specifically limited and examples include bacteria and yeasts. The host microorganism is preferably Escherichia coli. When the enzyme is a natural protein, bacteria expressing an endogenous HPAH can be used instead of the recombinant microorganism as described above. FIG. 1(B) shows a mechanism of enzymatic conversion activity in the bacterial reaction system.

When a microorganism is used for the conversion of the compounds, compound-conversion efficiency increases when a surfactant is added to a medium. The type of the surfactant to be used is not specifically limited and examples include SDS, Triton X-100, and Tween 80. The concentration of the surfactant is not also specifically limited and those skilled in the art can choose an appropriate concentration. The concentration is preferably from about 0.1% to about 10%, both inclusive, more preferably from about 0.5% to about 2%, both inclusive, and most preferably about 1%.

For the conversion of the compounds, an extract of a bacterium expressing an endogenous HPAH or a recombinant microorganism, or HPAH purified from a bacterium expressing an endogenous HPAH or a recombinant microorganism may be used rather than using a microorganism. The purification process is not specifically limited and may be crude purification or high-purity purification. When an extract or a purified HPAH is used, the compound can be converted in vitro by providing a reconstruction system with the addition of, for example, FAD or NAD (see, e.g., Biochemistry vol. 49, p. 372-385 (2010)).

### ==Method of Measuring Activity of Conversion of a Compound==

In one embodiment of the present invention, the enzymatic activity of conversion of the compound (I) to the compound (II) can be measured by administering a 4-hydroxyphenylacetate 3-hydroxylase (HPAH) analog to the compound (I) to detect the compound (II). The HPAH analog herein is a homolog of HpaB (SEQ ID NO.: 1) or HpaC (SEQ ID NO.: 2) derived from the Pseudomonas aeruginosa PAO1 strain and is a protein having an amino acid sequence which is at least 50%, 60%, 70%, 80%, or 90% homologous to HpaB or HpaC. It is thus possible to determine whether a given HPAH analog has the activity of conversion of the compound. The method of measuring the homology is not specifically limited and examples include BLAST, FASTA, and PSI-BLAST.

### ==Method of Screening Enzymes for Those Having Activity of Conversion of a Compound==

In one embodiment of the present invention, it is possible to screen 4-hydroxyphenylacetate 3-hydroxylase (HPAH) analogs for an enzyme converting the compound (I) to the compound (II) by administering a plurality of HPAH analogs to the compound (I), detecting the compound (II), and selecting the HPAH analog (s) for which the conversion has been detected. The HPAH analogs herein are proteins having an amino acid sequence which is at least 50%, 60%, 70%, 80%, or 90% homologous to HpaB or HpaC derived from the Pseudomonas aeruginosa PAO1 strain. It is thus possible to determine whether a given HPAH analog has the activity of conversion of the compound. The method of measuring the homology is not specifically limited and examples include BLAST, FASTA, and PSI-BLAST.

### EXAMPLES

### (1) Construction of Recombinant Escherichia coli Expressing Two-Component Flavin-Dependent Oxidase HpaBC

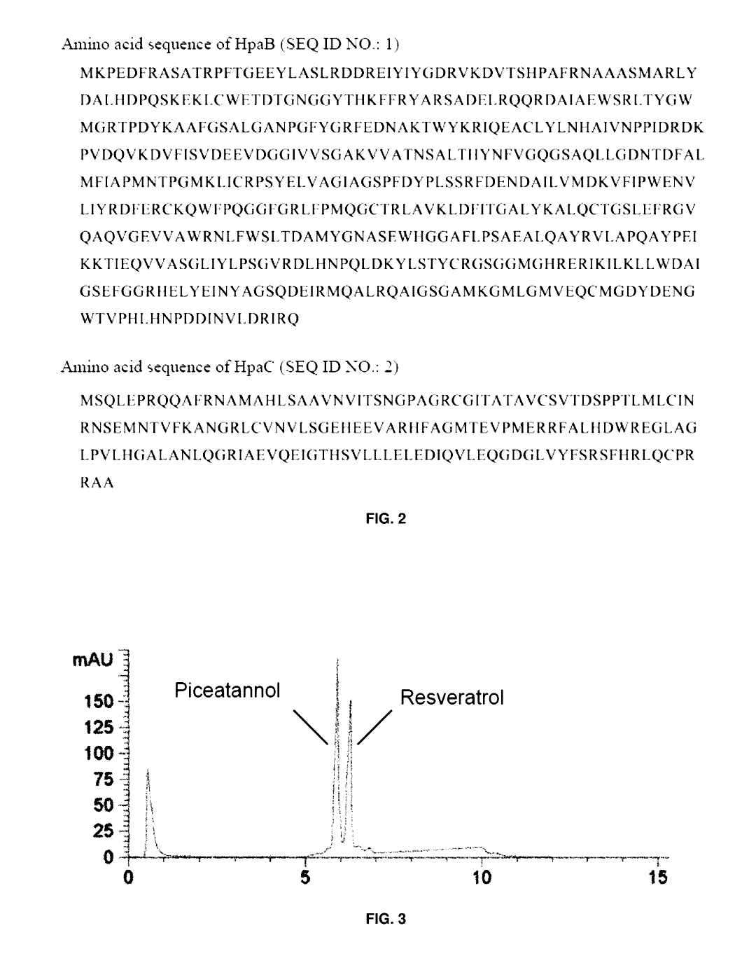

A gene that encodes the two-component flavin-dependent oxidase HpaBC of which amino acid sequence is shown in FIG. 2 was inserted into the pETDuet-1 vector (Novagen) and the recombinant vector was introduced into the Escherichia coli BL21 Star (DE3) strain (Invitrogen).

More specifically, the hpaB gene (ORF no. PA4091) that encodes a monooxygenase component and the hpaC gene (ORF no. PA4092) that encodes an oxide reductase component were amplified with PCR using genome of the Pseudomonas aeruginosa PAO1 strain (ATCC 47085D-5, GenBank Accession no. NC#002516) as template. Sense primer hpaB-s and antisense primer hpaB-as used for the amplification of the hpaB gene as well as sense primer hpaC-s and antisense primer hpaC-as used for the amplification of the hpaC gene are given below.

| (SEQ ID NO.: 3) | |
|---|---|
| hpaB-s: | TTCTCATGAAACCCGAAGATTTCCGTGCCT |
| (SEQ ID NO.: 4) | |
| hpaB-as: | CGCGGATCCTCATTGGCGGATGCGATCGAG |
| (SEQ ID NO.: 5) | |
| hpaC-s: | TTCCATATGTCCCAGCTCGAACCCAGGCAG |
| (SEQ ID NO.: 6) | |
| hpaC-as: | TTCGGTACCTCAGGCCGCCCGCCGGGGGCA |

After the amplification using PCR, both ends were digested with restriction enzymes BspHI and BamHI for the hpaB gene and NdeI and KpnI for the hpaC gene and then the respective DNAs were inserted into 1 and 2 of the multi-cloning site of pETDuet-1. The constructed plasmid (pETDhpaBC) was introduced into the E. coli BL21 Star (DE3) strain using electroporation.

### (2) Identification of a Conversion Product of Resveratrol, Produced by a HpaBC-Expressing Recombinant Escherichia coli

The constructed recombinant Escherichia coli was inoculated into LB medium (tryptone 1%, yeast extract 0.5%, NaCl 1%, pH 7.0) containing 100 μg/ml of ampicillin and incubated at 25° C. After 12 hours, 1 mM of isopropyl-β-D-thiogalactopyranoside was added and incubated for additional 12 hours to induce expression of the hpaBC gene. The bacteria were harvested, washed with 50 mM of potassium phosphate buffer (pH 7.5) containing 10% (v/v) of glycerol, and suspended in the same buffer. The suspension was used for the reaction with resveratrol; specifically, 100 μl of a reaction mixture consisting of 8 g/l (dry cell weight) of HpaBC-expressing recombinant Escherichia coli, 10 mM of resveratrol, 4% (v/v) of dimethylsulfoxide, 10% (v/v) of glycerol, and 50 mM of potassium phosphate buffer (pH 7.5) was prepared and shaken at 30° C. for 24 hours.

After the reaction, 10 μl of 5N HCl, 400 μl of H₂O, and 500 μl of methanol were added to prepare a sample for HPLC analysis. The HPLC analysis was performed using a system (1100 series) manufactured by Agilent Technologies and a column (XTerra MS C18 IS, length 4.6×20 mm, particle diameter: 3.5 μm) manufactured by Waters Corporation. For the developing solvent, 10 mM of acetonitrile/methanol/potassium phosphate buffer (pH 2.7)=2.5/2.5/95 (solvent A) and acetonitrile (solvent B) were used. The sample was eluted at a flow rate of 1 ml/min starting at 0% solvent B over 0-3 min, and then increasing the solvent B with a linear gradient to 70% over 3-12 min.

As a result, a new peak (5.9 min) was detected in addition to the peak of the resveratrol (6.3 min) (FIG. 3). The retention time of the peak was the same as the standard piceatannol, and its PDA spectrum was also identical with that of piceatannol.

Furthermore, NMR and LC-MS analyses were carried out and it was shown that the NMR spectrum and mass of the compound corresponding to the new peak were the same as those of piceatannol. Thus, the conversion product of resveratrol was identified as piceatannol. The results of the NMR and LC-MS analyses were given below.

¹H NMR (600 MHz, [D₆]DMSO): δ=6.13 (dd, J=1.8, 1.8 Hz, 1H), 6.39 (d, J=1.8 Hz, 2H), 6.73 (d, J=17.6 Hz, 1H), 6.73 (d, J=7.8 Hz, 1H), 6.84 (dd, J=7.8, 1.8 Hz, 1H), 6.86 (d, J=17.6 Hz, 1H), 6.98 (d, J=1.8 Hz, 1H); ¹³C NMR (600 MHz, [D₆]DMSO): 5=102.2, 104.8, 113.7, 116.2, 119.0, 126.0, 128.7, 129.1, 139.7, 145.8, 145.9, 158.9; MS (ESI) (m/z): calculated for C₁₄H₁₁O₄ [M-H]⁻, 243.1; found, 243.2.

As apparent from the above, resveratrol can be converted into piceatannol by administering the HpaBC-expressing recombinant Escherichia coli to resveratrol.

When the reaction was continued for a longer time, a new peak (5.4 min) was detected in addition to the peak of piceatannol in the HPLC analysis. LC-MS analysis was carried out and it was shown that a compound corresponding to this peak had [M-H]⁻ of 259.2 and was identified as a compound in which two oxygen atoms had been added to resveratrol. This indicated that resveratrol was converted to the following compound through piceatannol.

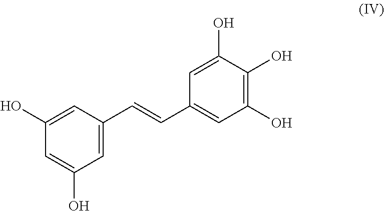 [SMILES: Oc1cc(O)cc(/C=C/c2cc(O)c(O)c(O)c2)c1]

### (3) Investigation of Conditions for Resveratrol Conversion

Effects of the concentrations of the substrate, the bacteria, and the surfactant on the resveratrol conversion activity were examined (Table 1). First, the bacterial cells (8 g/l in dry cell weight) were administered to 10 mM of the substrate under the same conditions as (2) to produce 4.3 mM of piceatannol in 24 hours (Entry 1). When the concentrations of the substrate and the bacteria were changed (Entries 2-5), the amount of the product piceatannol was increased to 8.8 mM (Entry 5). Furthermore, when the surfactant was added to the reaction mixture (Entries 6-9), the amount of the product was significantly increased at the concentration of 1% (v/v). In particular, in the presence of 1% (v/v) of Tween 80, 25 mM (6.1 g/l) of piceatannol was produced from 30 mM of resveratrol at a molar conversion yield of 83% (Entry 7). This is more than six times higher than the yield obtained when the HpaBC derived from Escherichia coli was used (Curr Opin Biotech vol. 26, p. 71-78 (2014)).

TABLE 1
Investigation of resveratrol conversion conditions
using HpaBC-expressing Escherichia coli

| | Substrate | Cells | Detergent | | Product | Conversion |
|---|---|---|---|---|---|---|
| Entry | (mM) | (g/l) ^{a} | (% v/v) | | (mM) | (%) ^{b} |
| 1 | 10 | 8 | 0 | | 4.3 | 43 |
| 2 | 10 | 16 | 0 | | 6.2 | 62 |
| 3 | 20 | 16 | 0 | | 7.2 | 36 |
| 4 | 20 | 32 | 0 | | 8.1 | 41 |
| 5 | 30 | 32 | 0 | | 8.8 | 29 |
| 6 | 30 | 32 | 0.1 | (Tween 80) | 13 | 43 |
| 7 | 30 | 32 | 1 | (Tween 80) | 25 | 83 |
| 8 | 30 | 32 | 0.1 | (TritonX-100) | 9.9 | 33 |
| 9 | 30 | 32 | 1 | (TritonX-100) | 22 | 73 |

^{a} Dry well weight/liter
^{b} Conversion (%) = Product (mM)/Substrate (mM) × 100

### (4) Synthesis of Piceatannol on a Flask Scale

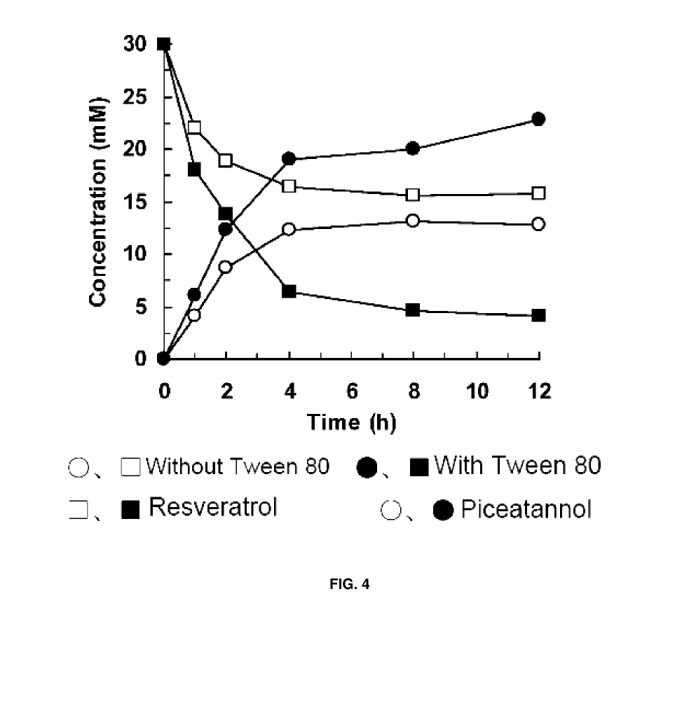

Piceatannol was synthesized on a flask scale under the optimum reaction condition determined in (3); more specifically, 20 ml of reaction mixture consisting of 32 g/l (dry cell weight) of HpaBC-expressing recombinant Escherichia coli cells, 30 mM of resveratrol, 4% (v/v) of dimethyl sulfoxide, 10% (v/v) of glycerol, and 50 mM of potassium phosphate buffer (pH 7.5) was prepared and shaken at 30° C. As a result, 13 mM (2.9 g/l) of piceatannol was produced in 12 hours without addition of the surfactant Tween 80 whereas 23 mM (5.2 g/l) of piceatannol was produced in the presence of 1% (v/v) of Tween 80 (FIG. 4). As apparent from the above, piceatannol could be also synthesized in an efficient manner on a flask scale.

### INDUSTRIAL APPLICABILITY

The present invention makes it possible to provide novel methods of producing hydroxystilbene.

1. A method of producing a compound having the general formula (II): 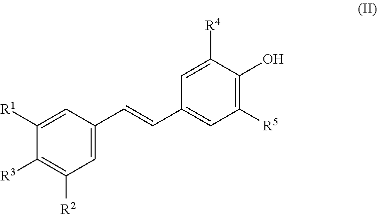
wherein
R¹ to R³ are each —H, —OH or —OR;
R is independently selected from a C1-6 alkyl group and a glycosyl group; and
one of R⁴ and R⁵ is —H and the other is —OH,
comprising the step of administering 4-hydroxyphenylacetate 3-hydroxylase (HPAH) to a compound having the general formula (I): 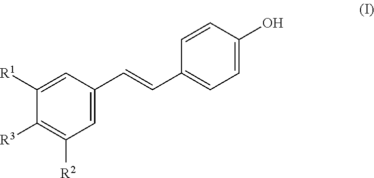
wherein
R¹ to R³ are each —H, —OH or —OR; and
R is independently selected from a C1-6 alkyl group and a glycosyl group.

2. A method of producing a compound having the general formula (III): 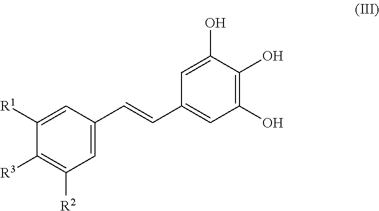
wherein
R¹ to R³ are each —H, —OH or —OR; and
R is independently selected from a C1-6 alkyl group and a glycosyl group,
comprising the step of administering 4-hydroxyphenylacetate 3-hydroxylase (HPAH) to a compound having the general formula (I): 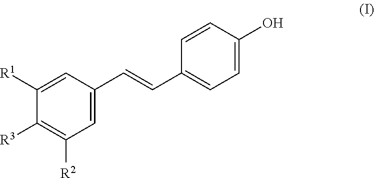
wherein
R¹ to R³ are each —H, —OH or —OR; and
R is independently selected from a C1-6 alkyl group and a glycosyl group,
and/or a compound having the general formula (II): 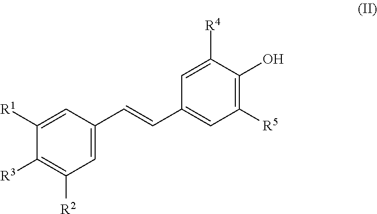
wherein
R¹ to R³ are each —H, —OH or —OR;
R is independently selected from a C1-6 alkyl group and a glycosyl group; and
one of R⁴ and R⁵ is —H and the other is —OH.

3. The method according to claim 1, wherein R³ is —H.

4. The method according to claim 3, wherein the compound having the general formula (II) is piceatannol.

5. The method according to claim 1, wherein the HPAH is a recombinant protein or a natural protein.

6. The method according to claim 1, wherein at least one selected from the group consisting of a purified HPAH recombinant protein, a purified HPAH natural protein, an extract from a bacterium expressing an endogenous HPAH, an extract from a microorganism expressing an exogenous HPAH, a bacterium expressing an endogenous HPAH, and a microorganism expressing an exogenous HPAH is administered to the compound that is a substrate.

7.-8. (canceled)

9. The method according to claim 6, wherein the bacterium expressing an endogenous HPAH or the microorganism expressing an exogenous HPAH is administered to the compound in the presence of a surfactant.

10.-13. (canceled)

14. The method according to claim 2, wherein R³ is —H.

15. The method according to claim 14, wherein the compound having the general formula (II) is piceatannol.

16. The method according to claim 14, wherein the HPAH is a recombinant protein or a natural protein.

17. The method according to claim 14, wherein at least one selected from the group consisting of a purified HPAH recombinant protein, a purified HPAH natural protein, an extract from a bacterium expressing an endogenous HPAH, an extract from a microorganism expressing an exogenous HPAH, a bacterium expressing an endogenous HPAH and a microorganism expressing an exogenous HPAH is administered to the compound that is a substrate.

18. The method according to claim 17, wherein the bacterium expressing an endogenous HPAH or the microorganism expressing an exogenous HPAH is administered to the compound in the presence of a surfactant.
